(19) United States
(12) Patent Application Publication
(10) Pub. No.: US 2021/0346445 A1
(43) Pub. Date: Nov. 11, 2021

(54) INFUSED CANNABIS PRODUCT SYSTEM AND METHOD

(71) Applicant: Peter Michael Peterson, Spokane, WA (US)

(72) Inventor: Peter Michael Peterson, Spokane, WA (US)

(21) Appl. No.: 16/870,615

(22) Filed: May 8, 2020

(51) Int. Cl. A61K 36/185; B29C 64/141; B29C 64/314; B65B 1/30

(52) U.S. Cl. CPC A61K 36/185; B29C 64/141; B33Y 10/00; B65B 1/30; B29C 64/314

(57) ABSTRACT

A method of producing a cannabis product includes loading and initiating materials the materials including cannabis materials. The method also includes sealing the environment. The method also includes fragmenting the materials by a fragmentation machine. Further the method includes binding, shaping, and portioning the materials.

### BACKGROUND

The statements in this section merely provide background information related to the present disclosure and may not constitute prior art.

Cannabis has long been used for both medicinal and recreational purposes. In both cases, there has always been a need for a more consistent product with improved and consistent flavor profiles.

Conventionally, the only non-invasive way to adjust cannabinoid and terpene profiles has been through growing and breeding. Another conventional method of attempting to achieve consistent results is to do infused mixes/blends. However, the only vehicle for delivery of these infused mixes/blends is infused joints. The process of producing these infused mixes/blends is far from scientific or consistent and therefore the end product varies wildly. The only variable that processors attempt to control for in the processing of these infused mixes/blends is the total THC level. Even when attempting to control the THC level, the results still vary significantly from batch to batch.

Accordingly, there is a need for systems, methods, and equipment which may be used to produce infused cannabis products with consistent results and more desired performance and flavor profiles.

### BRIEF SUMMARY

In an illustrative embodiment, a method of producing a cannabis product includes loading and initiating materials the materials including cannabis materials. The method also includes sealing the environment. The method also includes fragmenting the materials by a fragmentation machine. Further the method includes binding, shaping, and portioning the materials.

In another illustrative embodiment, a method of producing a cannabis product includes loading and initiating materials, the materials including cannabis materials. The method also includes pre-processing the materials. Further, the method includes sealing the environment. Further still, the method includes fragmenting the materials by a fragmentation machine and binding, shaping, and portioning the materials.

In another illustrative embodiment, a vehicle includes a method of producing a cannabis product includes loading and initiating materials the materials including cannabis materials and sealing the environment. The method also includes fragmenting the materials by a fragmentation machine and post-processing the materials by providing a second group of materials. Further, the method includes binding, shaping, and portioning the materials.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Illustrative embodiments are illustrated in referenced figures of the drawings. It is intended that the embodiments and figures disclosed herein are to be considered illustrative rather than restrictive.

FIG. 1 is an illustrative block diagram of a prep stage for processing.

FIG. 2 is an illustrative block diagram of a fragmenting stage for processing.

FIG. 3 is an illustrative block diagram of binding, shaping, and portioning for processing.

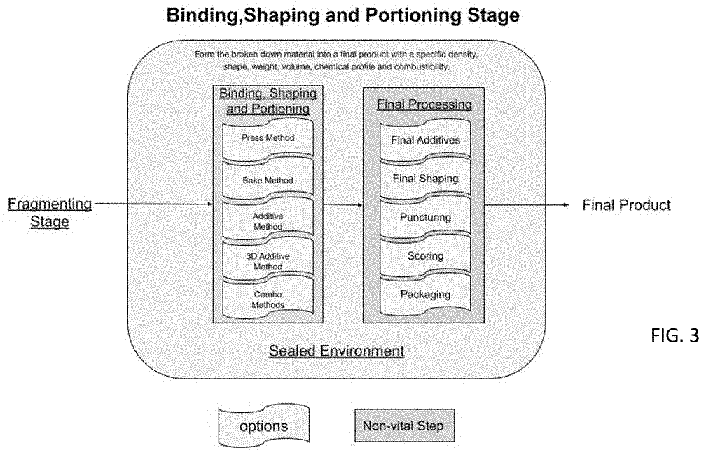

Like reference symbols in the various drawings generally indicate like elements.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

A system and method is described for producing infused cannabis products with desirable characteristics, such as but not limited to desirable and consistent flavor profiles, consistent THC levels, consistent burn performance, wide range of flavor profiles, etc. To produce such results, much attention must be paid to the processing of the cannabis products. The handling of cannabis in an uncontrolled environment exposes the material to unwanted chemical changes, for example light/UV damage, heat damage, physical damage, and mold spores. This environmental damage can ruin even the best quality material. Much of the unwanted aging of the product occurs in processing, where flower or bud is weighed, changed into other products and packaged. Prior to this it is likely the product has been stored or cured in a sealed environment.

An example of an activity where processing adversely impacts the starting material is making pre-roll joints. This activity speeds up the aging/spoiling process as the original flower is broken down (often in a grinder), and left exposed while the manual process of mixing, batching and packing the joints with the ground material occurs. By increasing the surface area of the original product through grinding or other methods, the process of spoiling increases environmental damage by at least an order of magnitude. This is then exacerbated as the grind itself is often stored in an unideal manner. The end product simply cannot match the quality of the input product. Hence, joints or pre-rolls are known on the consumer side to be an inferior product to flower(bud), even if the initial starting product is identical. Specifically, taste is degraded, harshness is increased and total cannabinoids are lowered.

In accordance with illustrative embodiments, the disclosed process is to take the input flower put it into the machine, seal the environment, change the internal environment in a beneficial manner for whatever process, manipulate the material in a desired manner and then seal the product via additive, and optionally package it or seal it in bulk for storage. These procedures will act to preserve or even increase the original quality of the input material.

In accordance with an exemplary embodiment the process may be broken down into three phases, (1) a prep stage, (2) a fragmenting stage, and (3) a binding, shaping, and portioning stage.

### Prep Stage

Loading and Initiating

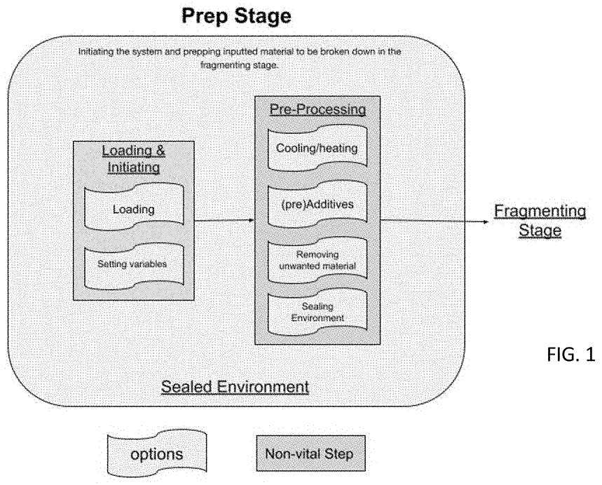

Referring now to FIG. 1, the product may first be loaded into a processing machine and the process initiated by sealing the environment for the steps that follow. In this step, options will be set on the type of input material: flower(bud), trim, or a mixture. Options for pre-processing will also be set, such as but not limited to the amount and type of additives, temperature settings, etc. This step may optionally occur at a later stage in the process with the fragmenting step in some embodiments.

After loading the system, the system is initiated and the necessary variables/controls would be set. These variables and controls include but are not limited to

1. Sealing the environment

2. Environment Settings

3. Temperature Settings

4. Additive Settings

5. Output Settings

6. Time Settings

Pre-Processing

In accordance with illustrative embodiments, Pre-Processing in the Prep Stage is optional. There are however reasons to pre-process the material such as but not limited to heating or cooling the product to an ideal temp, adjusting moisture levels, shaking or tumbling the product to remove free particles or unwanted material introduced in the harvest process, or coating the material with additives. Coating the product with additives like preservatives, humectants, flavor enhancers, cannabinoids, or substances to act as a lubricant or moisturizer during fragmentation may be desirable. Some or all of this activity may also occur in the post-processing of the Fragmenting Stage, but in some instances there may be benefits to doing so before the raw material is physically broken down.

After the material is loaded and the necessary variables are initiated the material would be passed to a rotary blender for removing unwanted material and inputting additives before it passes on to the Fragmenting Stage.

A Rotary Drum Blender/Mixer may be used for the following tasks in the Pre-Processing stage such as but not limited to:

1. Removal of unwanted particles and material

2. Introduction of additives
1. Preservatives
2. Enhancers
3. Lubricants
4. Cannabinoids
5. Moisturizers
6. Binders

3. any other type of material prep for fragmenting.

### Fragmenting Stage

Fragmenting Process

The Fragmenting Process, in its most basic form, is taking the original cannabis material, and grinding, shredding or cutting it down into smaller pieces. The effectiveness of this process and its implementation will depend heavily on the strategy taken in the Prep Stage. The pre-processing in the Prep Stage, particularly the additives inputted and temperature will influence how the material is fragmented. The Fragmenting Stage will then influence how easy it is to manipulate in the BSP stage. The characteristics of the output “grind” along with any other additives used in post-processing of the Fragmenting Stage, will likely be the greatest determiner of the quality of the end product.

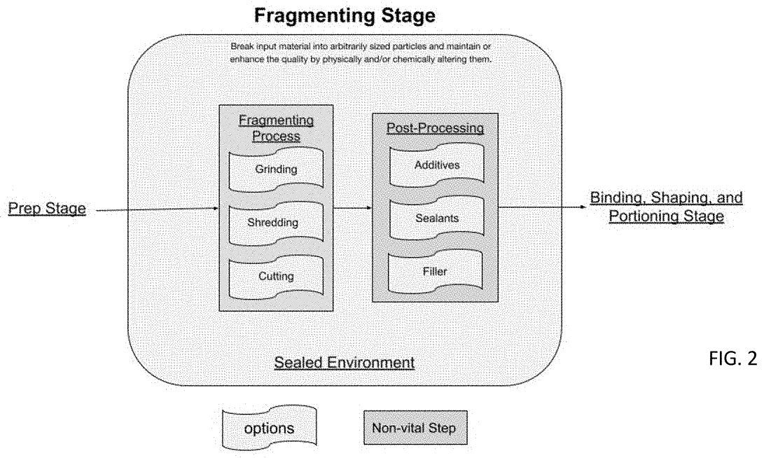

In accordance with illustrative embodiments as depicted in FIG. 2, the fragmenting process may be carried out in any of a variety of ways including but not limited to first sealing the environment and then fragmenting the materials using any of a grinder, a shredder, a cutter or slicer, or other similar means.

Once the materials have been fragmented, the process proceeds to the Post-Processing step where additives may be introduced that preserve freshness, modify flavor profile, adjust potency and aid in the Binding, Shaping and Portioning(BSP) Stage.

This step is similar to pre-processing in the Prep Stage. The ground or fragmented material is introduced to a rotary drum blender or mixer where unwanted particles (or kief) are removed with the rotary drum and then additives are then mixed in. The additives that may be introduced include but are not limited to Preservatives, Enhancers, Lubricants, Moisturizers, or Binders.

### Binding, Shaping and Portioning (BSP Process)

The BSP stage is a main part of the entire process. The BSP process will take the grind from the Fragmenting stage and manipulate it into an amalgamation of cannabis, cannabis extract, binders, fillers and enhancers such that the output is of a specific density, weight, shape, potency, combustibility, terpene and cannabinoid profile.

In its simplest form it is an infused product that can be smoked like raw flower(bud), i.e. in a pipe or a bong. This may be referred to as “infused flower”. The major flaw in the infused joint making process is how the material is handled and broken down. There are no tools used to allow the fragmenting to occur without oxidation, terpene loss, uv damage, etc., either during fragmenting, or after, in the production of the end product.

In accordance with an illustrative embodiment, the goal is to create an “infused flower” through the methods and strategies outlined below while attempting to maintain freshness, quality, and minimizing environmental damage which is vital to creating a product that consumers view as comparable to quality flower(bud). Packaged flower is still the largest subsector of the cannabis market, however infused products produced with the illustrative processes may produce a better alternative.

Currently the only non-invasive way to adjust cannabinoid and terpene profiles is through growing and breeding. Another avenue is to do infused mixes/blends. Currently the only vehicle for delivery of these blends is infused joints. This process is far from scientific or consistent and therefore the end product varies wildly. The only variable that Processors try to control for is the Total THC level, which still varies significantly from batch to batch.

By controlling the environment, blending materials with specific amounts of additives and forming the mix into a specific quantity and shape, an improved infused product may be achieved. There are a variety of BSP methods that may be applied without departing from the scope of the invention. These approaches include but are not limited to the methods listed and detailed below.

Mold & Press Method

The press method may be the simplest, most straightforward method. Here the fragmented product is delivered into an array of molds with a 2d or 3D injector arm and delivered in preset amounts, 1 g for example. In this method the pre-processing of the material would be vital to introducing any and all additives to the granulated material. Then the material is simply dropped/injected into the mold and pressed under a specific pressure, temperature and over a specific time to shape and bind everything together. The product would then be removed and post processed where a sealant or protectant is sprayed over each molded item and then may be further treated to ensure shelf life and quality.

Mold & Bake Method

This method is identical to the above method except instead of using a press and a dye to bind and form the end product, it would be “baked” at low temperatures over a relatively long time. A baking or freezing method might be useful in the final processing phase with other methods for binding, shaping and portioning.

Additive Method(Sheets)

This method involves repeated layering of material and binder or other additives on a plane. The granular material is laid down evenly, in a relatively thin layer, across the entire plane and then a set of inline spray nozzles introduces the additives and binders. These two steps are repeated until the desired thickness is reached. The result is a laminated sheet, either in this stage or in final processing, the layered product has tiny holes punched in it to make it burn more easily and more evenly. Another step that may occur here or in final processing is cutting the material into the desired size/shape.

3D Additive Method

This method involves a setup almost identical to a 3d printer with two main nozzles: one for granulated material and one for binders. Material is built up with one nozzle and binders and enhancers are progressively added. This method not only allows for arbitrary shapes but for binders and enhancers to be added in more specific places. The impetus would be to add binders in more strategic locations to provide an end product with structural integrity without making it too dense.

Combo Method

There are many permutations of the various elements listed above and many more not listed that remain within the scope of the invention. A combination of methods listed may also be used. For example, combinations of methods may include but are not limited to the following.

Additive Method with Mold & Press

In this method a single sheet of granulated material is laid out on a plane with no binders or additives. Then a matrix of top-down molds press from above and the material is infused simultaneously. In some embodiments, the materials may be deposited on a tray for post processing while any unused raw material would be swept up and used in following iterations.

Inline 3D Additive Method

This method is the additive method along an assembly line of sprayers, injectors and components to deposit the fragmented material. Product may be deposited and progressively built up along the line while binders and enhancers are introduced at various stages.

Final Processing

The complexity of final processing depends highly on what methods are used upstream in the entire process. In some embodiments the product may be shaped at the end, holes are created to increase combustibility or particular additives and “sealants” may also be introduced at this stage. Other possibilities such as heating or cooling are also optional. At this stage the product is “polished” for final packaging or storage. Final processing methods include but are not limited to the following methods.
1. Puncturing: putting holes in the product to increase its combustibility and make it burn evenly
2. Cutting, Shaping, Portioning: If the product has not been shaped or cut into the final shape with the desired weight it may be done here.
3. Coating: spraying the product with enhancers.
   1. Coating with cannabinoids
   2. Coating with humectants
   3. Coating with preservatives
   4. Coating with terpenes
   5. other
4. Heating or Baking: Heating or baking may be used to “set” or sinter binders and additives used in earlier stages.
5. Cooling or Freezing: If the product has required heating at another stage it may be required to cool it or freeze it to make it workable or to store it.
6. Package for storage
7. Package for retail

### Sealed Environment

As previously discussed, the handling of cannabis in an uncontrolled environment exposes the material to unwanted chemical changes, light/UV damage, heat damage, physical damage, and mold spores. This environmental damage can ruin even the best quality material. Much of the unwanted aging of the product occurs in Processing, where flower or bud is weighed, changed into other products and packaged. Prior to this it is likely the product has been stored or cured in a sealed environment.

While it may be desirable to have the entire process, from start to finish, done in a controlled environment it may not be necessary for all stages. Also, it may not be economical or plausible from the beginning of product development.

1. Prep Stage

It would be desirable to have the environment sealed at this point, but not necessary. Material at this stage is in its raw form and has not been broken down. Likely it has been cured and has been stored in a sealed environment It varies from company to company how they choose to store their product, some vacuum seal product in 5 lb bags, transparent or opaque, some use opaque, sealed buckets, it is likely some even still use mason jars. Nitrogen may or may not be used. It is often the case that the material is repeatedly exposed to the elements as arbitrary amounts are taken out to fill orders and then the product is resealed.

Due to the above reasons, it is less important to have a sealed environment at this point. The best reason to have a sealed environment at this stage would be if there were additives that required certain conditions to be applied and/or to be effective after they were applied. Also, this stage is the least invasive with respect to physically or chemically altering the product.

2. Fragmenting Stage

If we were forced to pick only one stage in which to provide a sealed environment, the Fragmenting Stage would be it, as this is where we expose the product to the most physical and environmental damage as well as where the most chemical changes would occur, either through exposure to the environment or the application of additives. The sealing process would involve vacuum sealing and/or displacing the air with an inert gas. Later in the post-processing phase of fragmenting, the “grind” can be sealed with preservatives, humectants and other additives in order to maintain freshness, limit oxidation, UV and mold damage.

The Fragmenting Stage may produce material that has been treated/sealed so that freshness is locked in and it is protected from environmental damage and chemical changes. Then if sealing of the environment throughout the remaining stages is not possible, quality has been maintained and freshness maintained to this stage.

Again, having the environment sealed at this stage may be important as breaking down the material increases the rate of chemical alteration and exposure to damage; the more surface area the more potential damage.

3. Binding, Shaping and Portioning Stage(BSP)

If the material has been “sealed” in the Fragmenting Stage and the methods we use in the BSP stage are gentle, it may or may not be necessary to have the environment sealed. However, the BSP Stage is a close second to the Fragmenting Stage in terms of importance, if not on a par, as manipulating the material and introducing additives will be benefitted by occurring in a closed environment.

The BSP processes will likely require chemicals and binders to form the desired end product, it may also cause physical damage to the “grind”, for example if we press it in a mold. All reasons to keep things sealed and controlled.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

1. A method of producing a cannabis product, comprising:
loading materials into a closable environment, the materials including cannabis materials;
initiating the materials in the closeable environment;
sealing the environment;
fragmenting the materials by a fragmentation machine; and
binding, shaping, and portioning the materials.

2. The method of claim 1, wherein initiating the materials includes setting variables for the materials.

3. The method of claim 1, wherein the fragmentation machine includes a grinder.

4. The method of claim 1, wherein the fragmentation machine includes a shredder.

5. The method of claim 1, wherein fragmentation machine includes a cutter.

6. The method of claim 1, wherein a second group of materials includes additives.

7. The method of claim 1, wherein the second group of materials includes sealants.

8. The method of claim 1, wherein the second group of materials includes fillers.

9. The method of claim 1, wherein the binding, shaping, and portioning includes using a press method.

10. The method of claim 1, wherein the binding, shaping, and portioning includes using a bake method.

11. The method of claim 1, wherein the binding, shaping, and portioning includes using an additive method.

12. The method of claim 1, wherein the binding, shaping, and portioning includes using a 3D additive method.

13. The method of claim 1, wherein the binding, shaping, and portioning includes using a combination method.

14. The method of claim 1, further comprising: pre-processing the materials by cooling or heating.

15. The method of claim 1, further comprising: pre-processing the materials by removing unwanted material.

16. The method of claim 1, further comprising: post-processing the materials by providing a second group of materials.

17. The method of claim 1, further comprising: final processing the materials including puncturing.

18. The method of claim 1, further comprising: final processing the materials including scoring.

19. A method of producing a cannabis product, comprising:
loading and initiating materials the materials including cannabis materials;
pre-processing the materials;
sealing the environment;
fragmenting the materials by a fragmentation machine; and
binding, shaping, and portioning the materials.

20. A method of producing a cannabis product, comprising:
loading and initiating a first group of materials the materials including cannabis materials;
sealing the environment;
fragmenting the first group of materials by a fragmentation machine;
post-processing the first group of materials by providing a second group of materials; and
binding, shaping, and portioning a mixture of the first group of materials and the second group of materials.
